(19) United States
(12) Patent Application Publication
(10) Pub. No.: US 2023/0309478 A1
(43) Pub. Date: Oct. 5, 2023

(54) CULTURING METHOD AND CULTURING DEVICE

(71) Applicant: HONDA MOTOR CO., LTD., Tokyo (JP)

(72) Inventors: Minoru Goto, Wako-shi (JP); Nozomi Shiobara, Wako-shi (JP); Shohei Kinoshita, Wako-shi (JP); Mizuho Doi, Wako-shi (JP); Kenji Machida, Wako-shi (JP); Satoshi Shiozaki, Wako-shi (JP)

(21) Appl. No.: 18/127,694

(22) Filed: Mar. 29, 2023

(30) Foreign Application Priority Data
Mar. 31, 2022 (JP) 2022-059347

(51) Int. Cl. A01G 33/00; C12M 1/04; C12M 1/02

(52) U.S. Cl. CPC A01G 33/00; C12M 27/04; C12M 41/20

(57) ABSTRACT

A culturing device is equipped with an accommodation unit, and a convection generating unit provided in the accommodation unit. In the accommodation unit, microalgae are cultured while the convection generating unit generates convection in the culturing solution in the accommodation unit. During the culturing, a portion of the culturing solution is drawn in through collection pipes by the pump. Thereafter, the culturing solution that was drawn in is discharged from the collection pipes into the culturing solution inside the accommodation unit.

### CROSS-REFERENCE TO RELATED APPLICATIONS

This application is based upon and claims the benefit of priority from Japanese Patent Application No. 2022-059347 filed on Mar. 31, 2022, the contents of which are incorporated herein by reference.

### BACKGROUND OF THE INVENTION

### Field of the Invention

The present invention relates to a culturing method and a culturing device for culturing microalgae in a culturing solution.

### Description of the Related Art

Heretofore, efforts aimed at mitigating or reducing the impact of climate change have continued, and toward the realization thereof, research and development in relation to the reduction of carbon dioxide emissions are being carried out. From this point of view, attention has been focused on microalgae. This is because microalgae consume carbon dioxide through photosynthesis.

Accordingly, a culturing device for culturing microalgae is anticipated as a device that contributes to alleviating or reducing the impact of climate change.

A culturing solution and the microalgae are accommodated in an accommodation unit. A sufficient amount of a culturing gas used in culturing for carrying out photosynthesis of the microalgae is supplied to the accommodation unit. Although the culturing solution and the microalgae are subjected to diffusion to some extent by the culturing gas, the microalgae undergo settling or sedimentation within the culturing solution. When the microalgae undergo such settling, the amount of light received by the microalgae decreases. As a result, the amount of photosynthesis becomes inadequate. From this standpoint, as disclosed in JP 2018-537948 A, a gas used to prevent settling is intermittently supplied with respect to the culturing solution inside the accommodation unit. In the technique disclosed in JP 2018-537948 A, the gas used to prevent settling is supplied to the culturing solution once every 30 minutes, and thereby causes the microalgae that have settled to float or be suspended within the culturing solution.

### SUMMARY OF THE INVENTION

In the case that the gas used to prevent settling is supplied to the accommodation unit in addition to the culturing gas, it is necessary for a gas supplying unit to discharge a large amount of gas. For this reason, regardless of the fact that the gas used to prevent settling or sedimentation is supplied intermittently, the gas supplying unit must have an ability to supply a large amount of the gas used to prevent settling or sedimentation.

A gas supplying unit having such a capacity consumes a large amount of electrical power per unit time period. Accordingly, in performing culturing in this manner, it is not easy to reduce the running cost.

The present invention has the object of solving the aforementioned problems.

According to one aspect of the present invention, there is provided a culturing method for culturing microalgae in a culturing device equipped with an accommodation unit in which a culturing solution and the microalgae are accommodated, and a gas supplying unit configured to supply a gas to the accommodation unit, wherein, in the accommodation unit, there are provided a convection generating unit configured to cause convection to be generated in the culturing solution, and a collection pipe configured to collect the culturing solution from the accommodation unit, and a pump configured to be capable of drawing in and discharging the culturing solution through the collection pipe, wherein, during culturing of the microalgae, and while causing convection to be generated in the culturing solution in the convection generating unit, the culturing solution is collected through the collection pipe by the pump, and thereafter, the collected culturing solution is discharged through the collection pipe by the pump.

According to another aspect of the present invention, there is provided a culturing device configured to culture microalgae in a culturing solution, the culturing device comprising an accommodation unit in which the culturing solution and the microalgae are accommodated, a convection generating unit provided in the accommodation unit and configured to cause convection to be generated in the culturing solution, a collection pipe provided in the accommodation unit and configured to collect the culturing solution from the accommodation unit, a pump configured to be capable of drawing in and discharging the culturing solution through the collection pipe, and a control unit configured to control the pump, wherein, during culturing of the microalgae and while causing convection to be generated in the culturing solution in the convection generating unit, the control unit performs a control to collect the culturing solution through the collection pipe by the pump, and thereafter, to discharge the collected culturing solution through the collection pipe by the pump.

Convection is generated by the convection generating unit in the culturing solution inside the accommodation unit. Under this condition, the culturing solution inside the accommodation unit is drawn in by the collection pipe. Thereafter, the culturing solution is discharged from the collection pipe into the accommodation unit. By the culturing solution being discharged in this manner, the microalgae that have settled to the bottom part of the accommodation unit are suspended or made to float. The suspended microalgae flow along with the convection. Consequently, the microalgae undergo agitation.

By drawing in the culturing solution through the collection pipe in this manner, and thereafter discharging the culturing solution from the collection pipe, the settled microalgae can be subject to agitation.

Accordingly, there is no need to supply a large amount of gas from the gas supplying unit in order to agitate the microalgae. Therefore, it is possible to select a gas supplying unit having an appropriate capacity. In this instance, the appropriate capacity is a capacity that is capable of supplying a sufficient amount of gas for culturing the microalgae.

Stated otherwise, there is no need to select a gas supplying unit having an excessive capacity. More specifically, according to the present invention, it is possible to use a gas supplying unit that consumes a small amount of electrical power per unit time period.

Therefore, in performing culturing, it is possible to reduce the running cost.

The microalgae that have been agitated are inhibited from settling or coalescing. Accordingly, the light is incident on the microalgae substantially evenly. Further, since the culturing solution is also agitated, carbon dioxide becomes diffused throughout the entirety of the accommodation unit. Due to this reason, the microalgae actively carry out photosynthesis. Since a large amount of carbon dioxide becomes fixed in the microalgae by way of photosynthesis, such a feature can be expected to contribute to a mitigation or a reduction in the impact of climate change.

The above and other objects, features, and advantages of the present invention will become more apparent from the following description when taken in conjunction with the accompanying drawings, in which a preferred embodiment of the present invention is shown by way of illustrative example.

### BRIEF DESCRIPTION OF THE DRAWINGS

FIG. 1 is a schematic system diagram of a culturing device according to an embodiment of the present invention;

FIG. 2 is a partially enlarged cross-sectional view of the culturing device;

FIG. 3 is a schematic front view of a guide unit having a different shape from the guide unit shown in FIG. 2;

FIG. 4 is a schematic process flow diagram of a culturing method according to an embodiment of the present invention;

FIG. 5 is a schematic system diagram showing a state in which convection is taking place in a culturing solution inside an accommodation unit in the culturing device;

FIG. 6A is a partially enlarged cross-sectional view showing a state in which the culturing solution is drawn in by a collection pipe, and FIG. 6B is a partially enlarged cross-sectional view showing a state in which the culturing solution is discharged from the collection pipe;

FIG. 7 is a schematic system diagram showing a state in which convection is taking place in a culturing solution inside an accommodation unit in the culturing device according to an embodiment of the present invention; and

FIG. 8 is a schematic system diagram showing a state in which convection is taking place in a culturing solution inside an accommodation unit in the culturing device according to an embodiment of the present invention.

### DETAILED DESCRIPTION OF THE INVENTION

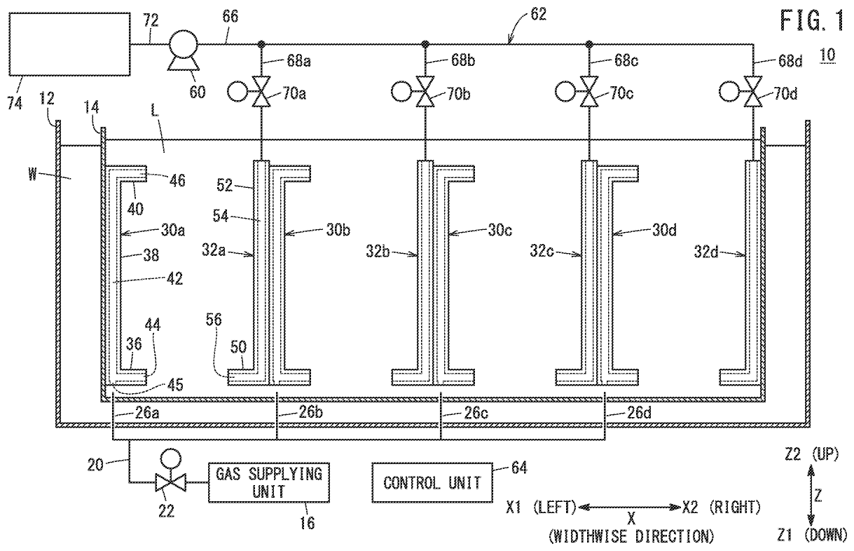

FIG. 1 is a schematic system diagram of a culturing device 10 according to a present embodiment. The culturing device 10 is equipped with a water storage unit 12 and an accommodation unit 14 that is accommodated in the water storage unit 12. The water storage unit 12 and the accommodation unit 14 are each formed respectively from a transparent/translucent material that allows light to pass through. Water W is stored in the water storage unit 12. The water W serves as a cooling solution that cools the culturing solution L inside the accommodation unit 14.

The culturing solution L and the microalgae are accommodated in the accommodation unit 14. As can be understood from this feature, the accommodation unit 14 serves as a culturing tank in order to culture the microalgae. The culturing solution L is typically water. Phosphorus, nitrogen, and potassium and the like are preferably added in advance to the culturing solution L. As specific examples of the microalgae, there may be cited the “Honda DREAMO strain” deposited at the National Institute for Product Evaluation Technology Patent Microorganism Depositary Center. In this instance, the National Institute for Product Evaluation Technology Patent Microorganism Depositary Center is located in Room 120, 2-5-8 Kazusa Kamatari, Kisarazu City, Chiba Prefecture. The date of deposit of the Honda DREAMO strain is Apr. 22, 2016, and the accession number is FERM BP-22306.

The culturing device 10 comprises a gas supply pipe 20 that guides a carbon dioxide containing gas supplied from a gas supplying unit 16 to the accommodation unit 14. The gas supplying unit 16, for example, is an air pump. Alternatively, the gas supplying unit 16 may be an exhaust line from a factory. A supply valve 22 is provided in the gas supply pipe 20.

A first distribution pipe 26a, a second distribution pipe 26b, a third distribution pipe 26c, and a fourth distribution pipe 26d branch away from a downstream end part of the gas supply pipe 20. The first distribution pipe 26a to the fourth distribution pipe 26d enter into the interior of the accommodation unit 14 from a bottom wall of the accommodation unit 14. Downstream end parts of the first distribution pipe 26a to the fourth distribution pipe 26d face toward lower end outer surfaces of later-described guide units (a first guide unit 30a to a fourth guide unit 30d).

A plurality of individual guide units and a plurality of individual collection pipes are provided in the interior of the accommodation unit 14. Hereinafter, in order to facilitate distinguishing between the plurality of individual guide units, the guide unit arranged in close proximity to a left side portion of the accommodation unit 14 in FIG. 1 will be referred to as a first guide unit 30a. The guide unit positioned to the right of the first guide unit 30a will be referred to as a second guide unit 30b. The guide unit positioned to the right of the second guide unit 30b will be referred to as a third guide unit 30c, and the guide unit positioned to the right of the third guide unit 30c will be referred to as a fourth guide unit 30d. Similarly, in order to facilitate distinguishing between the plurality of individual collection pipes, the collection pipe adjacent to the left of the second guide unit 30b will be referred to as a first collection pipe 32a. The collection pipe adjacent to the left of the third guide unit 30c will be referred to as a second collection pipe 32b. The collection pipe adjacent to the left of the fourth guide unit 30d will be referred to as a third collection pipe 32c, and the collection pipe arranged in close proximity to a right side portion of the accommodation unit 14 in FIG. 1 will be referred to as a fourth collection pipe 32d. The left side portion of the accommodation unit 14 is one end part of the accommodation unit 14, and the right side portion of the accommodation unit 14 is another end part of the accommodation unit 14.

The first guide unit 30a to the fourth guide unit 30d are supported in the water storage unit 12 via non-illustrated supporting members. Alternatively, the first guide unit 30a to the fourth guide unit 30d may be welded to an inner surface of the accommodation unit 14. In a similar manner, the first collection pipe 32a to the fourth collection pipe 32d are also supported in the water storage unit 12 via non-illustrated supporting members. Alternatively, the first collection pipe 32a to the fourth collection pipe 32d may be welded to an inner surface of the accommodation unit 14.

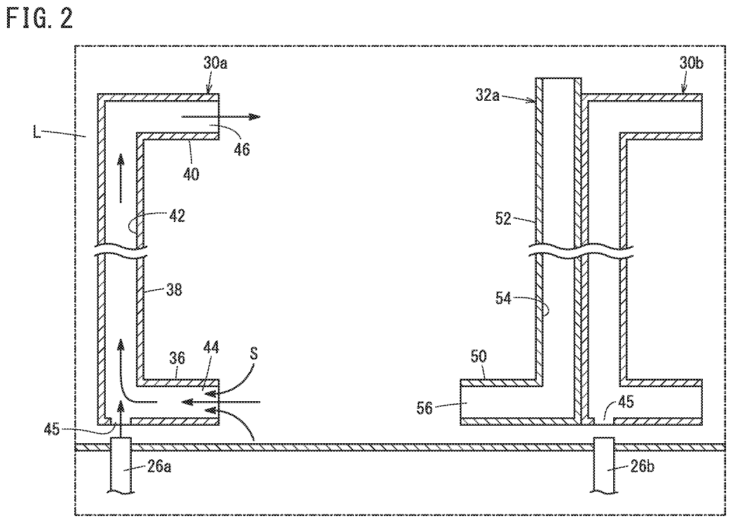

As shown in FIG. 2, the first guide unit 30a includes a suction pipe portion 36, a main body portion 38, and a discharge pipe portion 40. The main body portion 38 extends from a bottom part of the accommodation unit 14 toward the liquid surface of the culturing solution L. The direction in which the main body portion 38 extends, for example, is a vertical direction. When the accommodation unit 14 is inclined at a predetermined angle with respect to the vertical direction, the main body portion 38 is inclined at an angle corresponding to the angle of inclination of the accommodation unit 14. At a lower end of the main body portion 38, the suction pipe portion 36 projects out (in the X2 direction) toward the inner surface of the right side portion of the accommodation unit 14. At an upper end of the main body portion 38, the discharge pipe portion 40 projects out (in the X2 direction) toward the inner surface of the right side portion of the accommodation unit 14. The length of the suction pipe portion 36 is roughly equivalent to the length of the discharge pipe portion 40. Accordingly, the first guide unit 30a exhibits a sideways U-shape formed by the suction pipe portion 36, the main body portion 38, and the discharge pipe portion 40.

In this instance, the X direction is a horizontal direction (widthwise direction) that intersects with respect to the vertical direction of the accommodation unit 14. In FIG. 1, the widthwise direction is represented by the arrow X, and the vertical direction is represented by the arrow Z. In the arrow X, X1 indicates the left direction, which is one direction in the widthwise direction, and X2 indicates the right direction, which is a direction opposite to the direction of X1. In the arrow Z, Z1 indicates the lower direction, which is one direction in the vertical direction, and Z2 indicates an upper direction, which is a direction opposite to the direction of Z1.

The suction pipe portion 36, the main body portion 38, and the discharge pipe portion 40 are all hollow pipes. An internal space of the suction pipe portion 36 communicates with an internal space of the main body portion 38, and the internal space of the main body portion 38 communicates with an internal space of the discharge pipe portion 40. Due to the aforementioned communication, a guiding flow path 42 is formed in the interior of the first guide unit 30a. Since the first guide unit 30a exhibits a sideways U-shape, the guiding flow path 42 is an internal space also having a sideways U-shape. In the suction pipe portion 36, a portion thereof that faces (in the X2 direction) toward the inner surface of the right side portion of the accommodation unit 14 is open. Such an opening serves as a suction port 44 for drawing in the culturing solution L and the microalgae. A downstream end part of the first distribution pipe 26a faces toward a gas receiving port 45 that is formed in an outer surface of the lower end of the suction pipe portion 36.

In the discharge pipe portion 40 as well, a portion thereof that faces (in the X2 direction) toward the inner surface of the right side portion of the accommodation unit 14 is open. Such an opening serves as a discharge port 46 for discharging the gas and the culturing solution L. The gas discharged from a downstream end part of the first distribution pipe 26a flows into the guiding flow path 42 via the gas receiving port 45. The gas passes through the guiding flow path 42 and flows out into the culturing solution L from the discharge port 46.

The second guide unit 30b to the fourth guide unit 30d are configured in a similar manner to the first guide unit 30a. Accordingly, in the second guide unit 30b to the fourth guide unit 30d, the same reference numerals are used to indicate the same constituent elements as in the first guide unit 30a, and detailed description of such features will be omitted. Moreover, the suction port 44 of each of the first guide unit 30a to the fourth guide unit 30d faces (in the X2 direction) toward the inner surface of the right side portion of the accommodation unit 14. Also, the discharge port 46 of each of the first guide unit 30a to the fourth guide unit 30d faces (in the X2 direction) toward the inner surface of the right side portion of the accommodation unit 14.

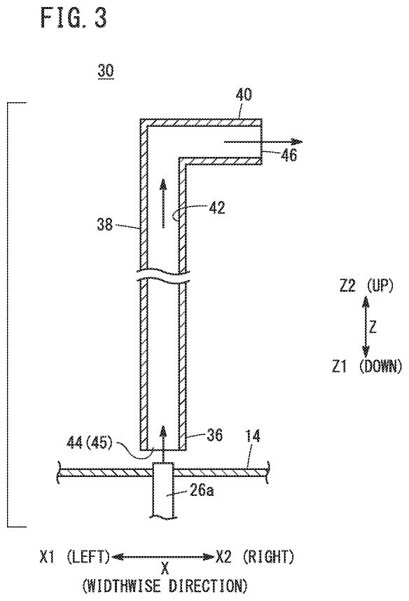

Moreover, as shown in FIG. 3, guide units 30 having an inverted L-shape formed by the suction pipe portion 36, the main body portion 38, and the discharge pipe portion 40 may be used. In this case, the suction pipe portion 36 extends downward (in the Z1 direction) of the accommodation unit 14 and faces toward a downstream end part of the first distribution pipe 26a. The gas supplied from the first distribution pipe 26a is drawn in from the suction port 44 together with the culturing solution L and the microalgae. Stated otherwise, in the guide units 30, the suction port 44 also functions as the gas receiving port 45.

Further, in the guide units 30, one end of the suction pipe portion 36, the main body portion 38, and the discharge pipe portion 40 may be open. In the suction pipe portion 36 and the main body portion 38, for example, the end parts thereof facing toward the X2 direction are opened. In the discharge pipe portion 40, for example, a lower end part thereof is opened. In this case, when the guide units 30 are viewed from below, the suction pipe portion 36 and the main body portion 38 exhibit a substantially U-shape that is opened in the X2 direction. In the suction pipe portion 36 and the main body portion 38, the end parts thereof facing toward the X2 direction are opened. In other words, the suction pipe portion 36 and the main body portion 38 are formed with U-shaped grooves that are opened in the Z1 direction and the X2 direction. In accordance with such a configuration, the U-shaped groove functions as the guiding flow path 42.

As shown in FIG. 2, the first collection pipe 32a includes a collection pipe portion 50 and a straight pipe portion 52. The straight pipe portion 52 extends substantially in parallel with the main body portion 38 of the first guide unit 30a. More specifically, when the main body portion 38 extends in the vertical direction, the straight pipe portion 52 also extends along the vertical direction. When the main body portion 38 is inclined at a predetermined angle with respect to the vertical direction, the straight pipe portion 52 is inclined at an angle corresponding to the angle of inclination of the main body portion 38. At a lower end of the straight pipe portion 52, the collection pipe portion 50 projects out (in the X1 direction) toward the inner surface of the left side portion of the accommodation unit 14.

Both the collection pipe portion 50 and the straight pipe portion 52 are hollow pipes. An internal space of the collection pipe portion 50 communicates with an internal space of the straight pipe portion 52. Due to communicating in this manner, a collection flow path 54 is formed in the interior of the first collection pipe 32a. In the collection pipe portion 50, a portion thereof that faces (in the X1 direction) toward the inner surface of the left side portion of the accommodation unit 14 is open. Such an opening serves as a collection port 56. As can be understood from this feature, the direction (the X1 direction) in which the collection port 56 of the first collection pipe 32a faces is opposite to the direction (the X2 direction) in which the discharge port 46 of the first guide unit 30a faces.

The second collection pipe 32b to the fourth collection pipe 32d are configured in a similar manner to the first collection pipe 32a. Accordingly, in the second collection pipe 32b to the fourth collection pipe 32d, the same reference numerals are used to indicate the same constituent elements as in the first collection pipe 32a, and detailed description of such features will be omitted. Moreover, the collection port 56 of each of the first collection pipe 32a to the fourth collection pipe 32d faces (in the X1 direction) toward the inner surface of the left side portion of the accommodation unit 14.

In the foregoing manner, the directions of the suction ports 44 and the discharge ports 46 of the first guide unit 30a to the fourth guide unit 30d are oriented in the X2 direction. In contrast thereto, the directions of the collection ports 56 of the first collection pipe 32a to the fourth collection pipe 32d are oriented in the X1 direction.

As shown in FIG. 1, the culturing device 10 includes a collection pump 60, a collection line 62, and a control unit 64.

The collection line 62 includes a main collection line 66, a first auxiliary collection line 68a, a second auxiliary collection line 68b, a third auxiliary collection line 68c, and a fourth auxiliary collection line 68d. The first auxiliary collection line 68a to the fourth auxiliary collection line 68d branch off from one end part of the main collection line 66. The other end of the main collection line 66 is connected to the collection pump 60. The first auxiliary collection line 68a to the fourth auxiliary collection line 68d are connected to upper ends of the straight pipe portions 52 of the first collection pipe 32a to the fourth collection pipe 32d. A first solenoid valve 70a, a second solenoid valve 70b, a third solenoid valve 70c, and a fourth solenoid valve 70d are provided respectively in the first auxiliary collection line 68a to the fourth auxiliary collection line 68d.

The collection pump 60 is a pump that is capable of performing a forward rotation operation and a reverse rotation operation. For example, the collection pump 60 draws in the culturing solution L into the accommodation unit 14 at a time when performing the forward rotation operation. In contrast thereto, the collection pump 60 discharges the culturing solution L into the accommodation unit 14 at a time when performing the reverse rotation operation. In this manner, the collection pump 60 is capable of drawing in the culturing solution L, and is also capable of discharging the culturing solution L.

The collection pump 60 is connected to a collection tank 74 via a liquid delivery pipe 72. The microalgae and the culturing solution L inside the accommodation unit 14 are drawn in into the first collection pipe 32a to the fourth collection pipe 32d by the forward rotation operation of the collection pump 60. Thereafter, the microalgae and the culturing solution L are delivered to the collection tank 74 through the liquid delivery pipe 72. More specifically, the microalgae and the culturing solution L are stored in the collection tank 74.

The control unit 64 is electrically connected to the supply valve 22, the collection pump 60, and the first solenoid valve 70a to the fourth solenoid valve 70d. The control unit 64 causes the collection pump 60 to be started or stopped, and causes the direction of rotation of the collection pump 60 to be reversed. The control unit 64 opens and closes the supply valve 22 and the first solenoid valve 70a to the fourth solenoid valve 70d. A predetermined time period is input to the control unit 64 in advance. The control unit 64 compares the predetermined time period with an elapsed time period from a point in time at which a later-described gas supply starting step S1 is executed.

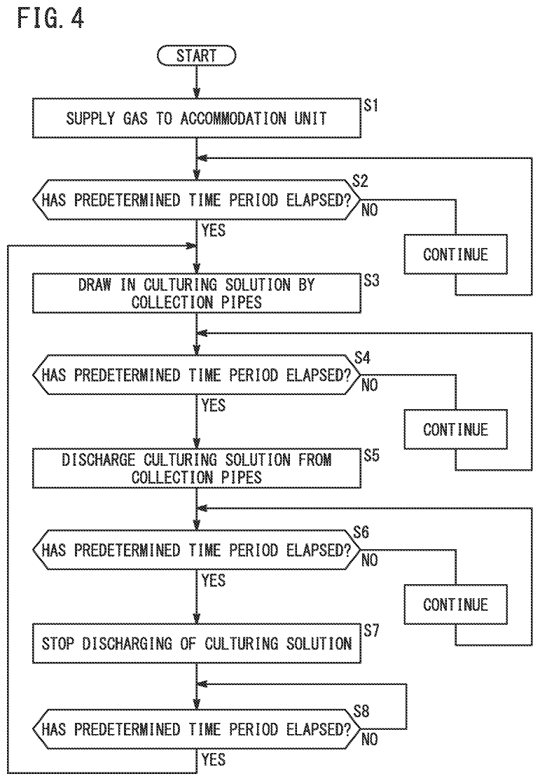

Next, with reference to the schematic process flow diagram shown in FIG. 4, a description will be given concerning the culturing method according to the present embodiment. Such a culturing method includes a gas supply starting step S1, a first determination step S2, a suction starting step S3, a second determination step S4, a discharge starting step S5, a third determination step S6, and a discharge stopping step S7. These steps S1 to S7 are executed based on a sequence control executed by the control unit 64.

The operator inputs a command signal to “execute the gas supply starting step S1” with respect to the control unit 64. Such a command signal is input to the control unit 64, for example, accompanying a switch provided in the control unit 64 being turned ON. In the case that the gas supplying unit 16 is an air pump, the control unit 64 which has received such a command signal initiates the air pump. Further, the control unit 64 opens the supply valve 22. Carbon dioxide is contained in the atmospheric air that is drawn in by the air pump. Carbon dioxide is similarly contained in the exhaust gas that is discharged from an exhaust line of a factory. Accordingly, the gas that flows through the gas supply pipe 20 is a carbon dioxide containing gas.

The carbon dioxide containing gas that has flowed through the gas supply pipe 20 is distributed to the first distribution pipe 26a to the fourth distribution pipe 26d. When a description is made taking the first distribution pipe 26a as an example, a downstream end part of the first distribution pipe 26a faces toward the gas receiving port 45. Accordingly, the carbon dioxide containing gas that has flowed through the first distribution pipe 26a flows into the interior of the gas receiving port 45 from the first distribution pipe 26a.

The carbon dioxide containing gas that has flowed out from the downstream end part of the first distribution pipe 26a rises along the guiding flow path 42 in the Z2 direction. During rising thereof, as indicated by the arrow S in FIG. 2, the culturing solution L and the microalgae surrounding the periphery of the suction port 44 are drawn in from the suction port 44 in the vicinity of the bottom part of the accommodation unit 14. The culturing solution L that was drawn in is accompanied by the carbon dioxide containing gas and rises along the guiding flow path 42 in the Z2 direction.

After the carbon dioxide-containing gas and the culturing solution L have risen along the guiding flow path 42 of the main body portion 38, the forward movement direction is changed to a direction along the discharge pipe portion 40. Thereafter, the carbon dioxide containing gas and the culturing solution L are discharged into the culturing solution L inside the accommodation unit 14 through the discharge port 46. The carbon dioxide containing gas and the culturing solution L that have passed through the guiding flow path 42 flow out to the upper portion of the accommodation unit 14.

The carbon dioxide containing gas and the culturing solution L discharged from the discharge port 46, after having advanced toward the first collection pipe 32a, then come into contact with the straight pipe portion 52 of the first collection pipe 32a. Since the straight pipe portion 52 of the first collection pipe 32a extends along the vertical direction of the accommodation unit 14, the carbon dioxide containing gas and the culturing solution L descend along the straight pipe portion 52.

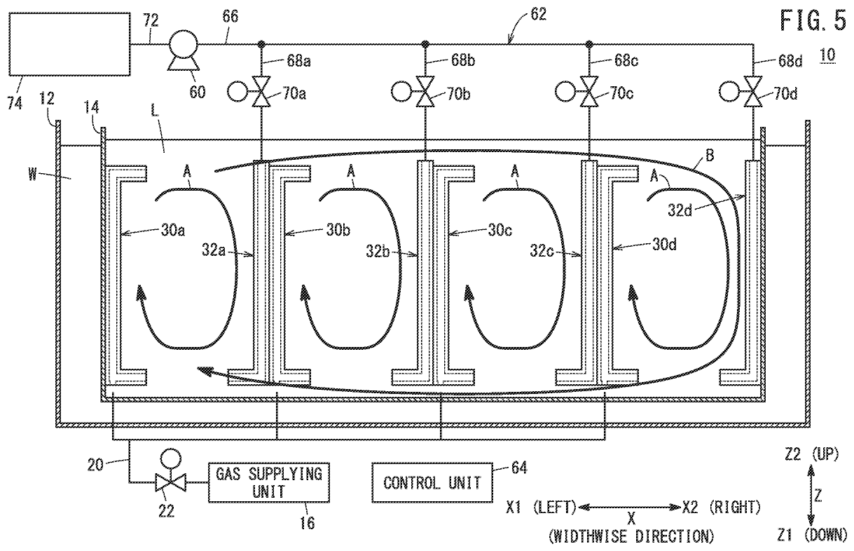

By the carbon dioxide containing gas and the culturing solution L advancing in the foregoing manner, as indicated by the arrow A in FIG. 5, a small amount of convection is generated between the first guide unit 30a and the first collection pipe 32a. Similarly, a small amount of convection is also generated between the second guide unit 30b and the second collection pipe 32b. A small amount of convection is also generated between the third guide unit 30c and the third collection pipe 32c. A small amount of convection is also generated between the fourth guide unit 30d and the fourth collection pipe 32d.

The suction ports 44 of the first guide unit 30a to the fourth guide unit 30d face mutually toward the same direction (the X2 direction). The discharge ports 46 of the first guide unit 30a to the fourth guide unit 30d also face mutually toward the same direction (the X2 direction). Further, the suction ports 44 and the discharge ports 46 are open toward the same direction. Therefore, four individual small amounts of convection are combined. As a result, as indicated by the arrow B, a large amount of convection is generated inside the accommodation unit 14. In the foregoing manner, the gas supplying unit 16 and the first guide unit 30a to the fourth guide unit 30d constitute a convection generating unit that causes convection to be generated.

By the small amounts of convection and the large amount of convection, the carbon dioxide containing gas becomes diffused. More specifically, the carbon dioxide diffuses throughout the culturing solution L, and sufficiently dissolves in the culturing solution L. Further, the microalgae are also agitated by the small amounts of convection and the large amount of convection. Therefore, the microalgae are prevented from settling or coalescing. For the aforementioned reasons, the microalgae are satisfactorily cultured throughout the entirety of the accommodation unit 14. During culturing thereof, the microalgae sufficiently fix the carbon dioxide based on photosynthesis being actively carried out. Consequently, the carbon dioxide is consumed.

As has been described above, based on the carbon dioxide being consumed by culturing the microalgae, it is possible to contribute to mitigating or reducing the impact of climate change. In the case that phosphorus, nitrogen, potassium, and the like are added to the culturing solution L, the microalgae take in such inorganic substances as nutrients.

As noted previously, in accordance with the carbon dioxide containing gas supplied to the culturing solution L via the gas supply pipe 20, the culturing solution L and the microalgae inside the accommodation unit 14 are agitated. However, there is a tendency for the microalgae to settle over time. In order to prevent the microalgae from settling, the control unit 64 performs a control in order to temporarily supply a large amount of the carbon dioxide containing gas with respect to the accommodation unit 14. Hereinafter, a description will be given in detail concerning this feature.

During a period in which culturing of the microalgae proceeds under a condition in which the small amounts of convection and the large amount of convection are generated in the manner described above, the control unit 64 executes the first determination step S2 while measuring an elapsed time period from the gas supply starting step S1 having been executed. In the first determination step S2, the control unit 64 compares the elapsed time period with the predetermined time period that was input to the control unit 64 in advance. In the case that the elapsed time period has not reached the predetermined time period (“NO” in step S2 in FIG. 4), the collection pump 60 is not started. Further, the first solenoid valve 70a to the fourth solenoid valve 70d are closed.

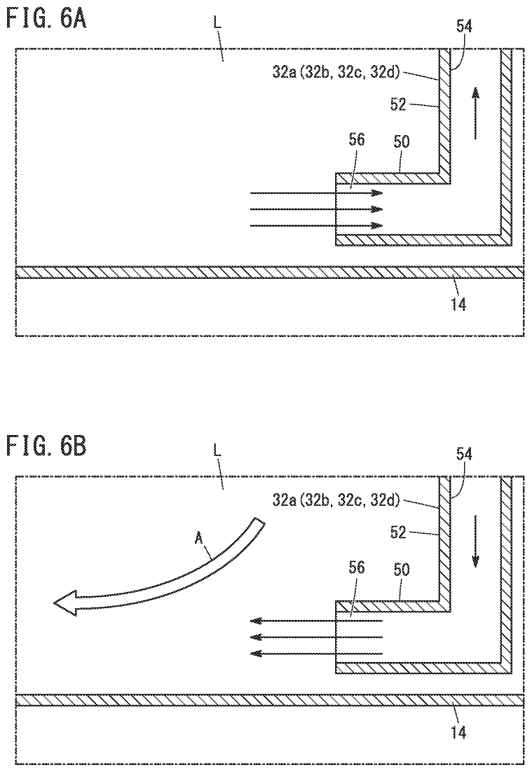

In contrast thereto, in the case that the elapsed time period has reached the predetermined time period (“YES” in step S2 in FIG. 4), the control unit 64 executes the suction starting step S3. Specifically, the control unit 64 starts the collection pump 60 to undergo forward rotation, and in addition, opens the first solenoid valve 70a to the fourth solenoid valve 70d. Consequently, the interior of the collection flow path 54 of the first collection pipe 32a to the fourth collection pipe 32d becomes under a negative pressure, and the culturing solution L in the accommodation unit 14 flows into the interior of the collection pipes (the collection flow path 54) through the collection port 56. Along with drawing in the culturing solution L in this manner, as shown schematically in FIG. 6A, the microalgae that have settled in the bottom part of the accommodation unit 14 float toward the collection port 56.

The control unit 64 executes the second determination step S4 while measuring the elapsed time period from having started the suction starting step S3. In the second determination step S4, the control unit 64 compares the elapsed time period with the predetermined time period that was input to the control unit 64 in advance. Moreover, it should be noted that the predetermined time period is sufficient, for example, for the collection flow path 54 of the first collection pipe 32a to the fourth collection pipe 32d to be filled with the culturing solution L and the microalgae. However, it is also possible to set the predetermined time period to an extent whereby the culturing solution L and the microalgae are stored at a predetermined amount in the collection tank 74.

In the case that the elapsed time period has not reached the predetermined time period (“NO” in step S4 in FIG. 4), the forward rotation operation of the collection pump 60 is continued. In contrast thereto, in the case that the elapsed time period has reached the predetermined time period (“YES” in step S4 in FIG. 4), the control unit 64 executes the discharge starting step S5. Specifically, the control unit 64 reverses the direction of rotation of the collection pump 60. Stated otherwise, the collection pump 60 performs the reverse rotation operation.

In accordance with this feature, as shown in FIG. 6B, the culturing solution L and the microalgae in the collection flow path 54 of the first collection pipe 32a to the fourth collection pipe 32d are discharged from the collection port 56. The microalgae that are still in a state of being settled on the bottom part of the accommodation unit 14 are pushed out by the culturing solution L and the microalgae that are discharged from the collection port 56. By being pushed out in this manner, the microalgae that have settled are suspended or made to float.

The suspended microalgae are made to flow along with the small amount of convection and the large amount of convection (refer to FIG. 5). Thereafter, the microalgae are drawn in from the suction ports 44 of the first guide unit 30a to the fourth guide unit 30d in the same manner as described above, and thereafter, the microalgae are discharged from the discharge ports 46 of the first guide unit 30a to the fourth guide unit 30d. Consequently, the microalgae having once settled on the bottom part of the accommodation unit 14 are agitated.

In this manner, according to the present embodiment, the culturing solution L and the microalgae inside the accommodation unit 14 are drawn into the first collection pipe 32a to the fourth collection pipe 32d, and thereafter, the culturing solution L and the microalgae that were drawn in are discharged into (returned to) the interior of the accommodation unit 14. Consequently, the microalgae that have settled on the bottom part of the accommodation unit 14 can be agitated along with the convection generated inside the accommodation unit 14. Therefore, since the rate at which light is received by the microalgae is improved, the photosynthesis of microalgae is activated. Therefore, the carbon dioxide can be consumed sufficiently.

Further, the aforementioned agitation is carried out by the collection pump 60 that is used in order to collect the culturing solution L and the microalgae. Accordingly, there is no need to provide a stirring blade and a motor or the like in the accommodation unit 14 in order to agitate the settled microalgae. Therefore, it is possible to reduce the size and scale of the culturing device 10, and to reduce the investment in equipment.

Further, it is not necessary to increase the amount of the gas supplied from the gas supplying unit 16, and thereby agitate the culturing solution L and the microalgae. As noted previously, this is because the culturing solution L and the microalgae can be subjected to agitation by operating the collection pump 60. More specifically, according to the present embodiment, the amount of gas supplied from the gas supplying unit 16 can be kept constant. Therefore, the gas supplying unit 16 is not required to have an excessive capacity in order to supply a large amount of gas to the accommodation unit 14. Accordingly, according to the present embodiment, it is possible to use a gas supplying unit 16 that consumes a small amount of electrical power per unit time period. Therefore, in performing culturing, it is possible to reduce the running cost.

The control unit 64 executes the third determination step S6 while measuring the elapsed time period from having started the discharge starting step S5. In the third determination step S6, the control unit 64 compares the elapsed time period with the predetermined time period that was input to the control unit 64 in advance. Moreover, the predetermined time period, for example, may be substantially equivalent to the time period from having started the suction starting step S3 until the start of the discharge starting step S5.

In the case that the elapsed time period has not reached the predetermined time period (“NO” in step S6 in FIG. 4), the reverse rotation operation of the collection pump 60 is continued. In contrast thereto, in the case that the elapsed time period has reached the predetermined time period (“YES” in step S6 in FIG. 4), the control unit 64 executes the discharge stopping step S7. Specifically, the control unit 64 closes the first solenoid valve 70a to the fourth solenoid valve 70d, and in addition, stops the collection pump 60. Consequently, discharging of the culturing solution L and the microalgae from the first collection pipe 32a to the fourth collection pipe 32d is stopped.

In the foregoing manner, according to the present embodiment, the culturing solution L and the microalgae are temporarily discharged into the accommodation unit 14 from the first collection pipe 32a to the fourth collection pipe 32d, and in accordance therewith, the culturing solution L and the microalgae inside the accommodation unit 14 can be agitated. Moreover, it should be noted that, even after the discharge stopping step S7 has been executed, the supply of the carbon dioxide containing gas from the gas supplying unit 16 to the accommodation unit 14 is continued. Stated otherwise, the microalgae continuously undergo culturing.

As shown in step S8 in FIG. 4, the control unit 64 measures an elapsed time period from when the discharge stopping step S7 was executed. In the case that the elapsed time period has reached the predetermined time period (“YES” in step S8 in FIG. 4), the control unit 64 sequentially executes the suction starting step S3, the second determination step S4, the discharge starting step S5, the third determination step S6, and the discharge stopping step S7. Thereafter, this cycle is repeated. After the culturing of the microalgae has been completed, the collection pump 60 performs the forward rotation operation, and the culturing solution L and the microalgae inside the accommodation unit 14 are collected in the collection tank 74.

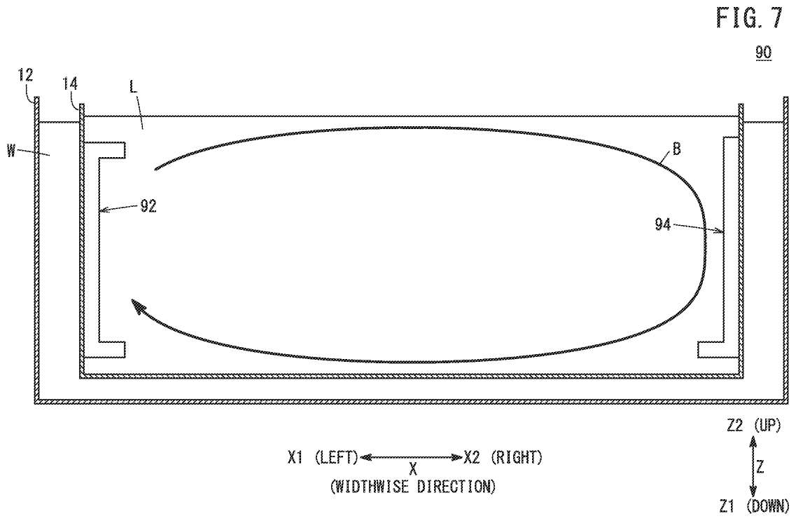

The number of the guide units and the collection pipes may be one each. FIG. 7 is a schematic system diagram of a culturing device 90 in accordance with such an aspect. Moreover, in FIG. 7, the gas supplying unit 16 and the gas supply pipe 20 and the like are omitted. Further, the water storage part 12, the accommodation unit 14, the water W, and the culturing solution L are designated by the same reference numerals as in FIG. 1.

The culturing device 90 is equipped with a single individual guide unit 92 and a single individual collection pipe 94. As shown in FIG. 7, the guide unit 92 is arranged on a left side portion (one end part) of the accommodation unit 14, and the collection pipe 94 is arranged on a right side portion (another end part) of the accommodation unit 14. In accordance with such a configuration, a large amount of convection (as shown by the arrow B) is generated between the guide unit 92 and the collection pipe 94. Accordingly, in such a culturing device 90 as well, the culturing solution L and the microalgae can be agitated in the same manner as in the culturing device 10.

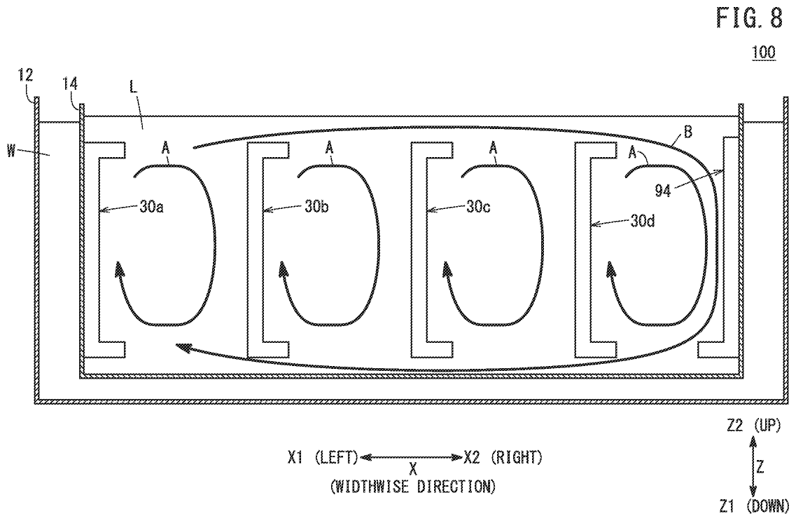

A single individual collection pipe may be provided with respect to the plurality of individual guide units. FIG. 8 is a schematic system diagram of a culturing device 100 in accordance with such an aspect. Moreover, in FIG. 8, the gas supplying unit 16 and the gas supply pipe 20 and the like are omitted. Further, the water storage unit 12, the accommodation unit 14, the water W, the culturing solution L, and the first guide unit 30a to the fourth guide unit 30d are designated by the same reference numerals as in FIG. 1. The single individual collection pipe 94 is designated by the same reference numeral as in FIG. 7.

The culturing device 100 is equipped with the first guide unit 30a to the fourth guide unit 30d, and a single individual collection pipe 94. As shown in FIG. 8, the first guide unit 30a is arranged on a left side portion (one end part) of the accommodation unit 14, and the collection pipe 94 is arranged on a right side portion (another end part) of the accommodation unit 14. The second guide unit 30b to the fourth guide unit 30d are arranged between the first guide unit 30a and the collection pipe 94. In accordance with such a configuration, the microalgae that have become settled in corners of the accommodation unit 14 where convection is unlikely to be generated are suspended or made to float by the first guide unit 30a and the collection pipe 94.

Further, the microalgae are agitated by the small amount of convection generated between the first guide unit 30a and the second guide unit 30b, the small amount of convection generated between the second guide unit 30b and the third guide unit 30c, the small amount of convection generated between the third guide unit 30c and the fourth guide unit 30d, and the small amount of convection generated between the fourth guide unit 30d and the collection pipe 94, and the large amount of convection generated between the first guide unit 30a and the unit pipe 94. Accordingly, in such a culturing device 100 as well, the culturing solution L and the microalgae can be agitated in the same manner as in the culturing device 10. In FIG. 8, the small amounts of convection are indicated by the arrows A, and the large amount of convection is indicated by the arrow B.

At a time when the environmental temperature around the periphery of the accommodation unit 14 is low, or at a time when the amount of light incident on the accommodation unit 14 is small, there is a tendency for the microalgae to settle. Accordingly, the steps from the suction starting step S3 to the discharge stopping step S7 may also be performed at a time when the temperature of the culturing solution L has become less than or equal to an allowable lower limit value, or when the amount of light incident on the accommodation unit 14 has become less than or equal to an allowable lower limit value. In this case, at least one of a temperature detector and a light quantity measuring instrument is provided in the culturing device.

As has been described above, in the present embodiment, there is disclosed the culturing method for culturing microalgae in the culturing device (10, 90, 100) equipped with the accommodation unit (14) in which the culturing solution (L) and the microalgae are accommodated, and the gas supplying unit (16) that supplies the gas to the accommodation unit, wherein, in the accommodation unit, there are provided the convection generating unit that causes convection to be generated in the culturing solution, and the collection pipe (32a to 32d, 94) that collects the culturing solution from the accommodation unit, and the pump (60) that is capable of drawing in and discharging the culturing solution through the collection pipe, wherein, during culturing of the microalgae, and while causing convection to be generated in the culturing solution in the convection generating unit, the culturing solution is collected through the collection pipe by the pump, and thereafter, the collected culturing solution is discharged through the collection pipe by the pump.

In the present embodiment, there is disclosed the culturing device (10, 90, 100) that cultures the microalgae in the culturing solution (L), the culturing device comprising the accommodation unit (14) in which the culturing solution and the microalgae are accommodated, the convection generating unit provided in the accommodation unit and that causes convection to be generated in the culturing solution, the collection pipe (32a to 32d, 94) provided in the accommodation unit and that collects the culturing solution from the accommodation unit, the pump (60) that is capable of drawing in and discharging the culturing solution through the collection pipe, and the control unit (64) that controls the pump, wherein, during culturing of the microalgae and while causing convection to be generated in the culturing solution in the convection generating unit, the control unit performs a control to collect the culturing solution through the collection pipe by the pump, and thereafter, to discharge the collected culturing solution through the collection pipe by the pump.

Convection is generated by the convection generating unit in the culturing solution inside the accommodation unit. Under this condition, the culturing solution inside the accommodation unit is drawn in by the collection pipe. Thereafter, the culturing solution is discharged from the collection pipe into the accommodation unit. By the culturing solution being discharged in this manner, the microalgae that have settled to the bottom part of the accommodation unit are suspended or made to float. The suspended microalgae flow along with the convection. Consequently, the microalgae undergo agitation.

Since the microalgae that have once been subjected to agitation are agitated in the manner described previously, it is not necessary to supply a gas from the gas supplying unit in order to agitate the culturing solution. More specifically, during the period that culturing is carried out, there is no need to increase the amount of gas supplied from the gas supplying unit. Therefore, there is no need to select a gas supplying unit having an excessive capacity. In the foregoing manner, according to the present embodiment, it is possible to select a gas supplying unit having an appropriate capacity.

Accordingly, it is possible to use a gas supplying unit that consumes a small amount of electrical power per unit time period. Therefore, in performing culturing, it is possible to reduce the running cost.

The microalgae that have been agitated are inhibited from settling or coalescing. Accordingly, the light is incident on the microalgae substantially evenly. Further, since the culturing solution is also agitated, carbon dioxide becomes diffused throughout the entirety of the accommodation unit. Due to this reason, the microalgae actively carry out photosynthesis. Since a large amount of carbon dioxide becomes fixed in the microalgae by way of photosynthesis, such a feature contributes to a mitigation or a reduction in the impact of climate change.

The convection generating unit includes, for example, the guide unit. More specifically, in the present embodiment, the culturing method is disclosed, in which the convection generating unit comprises the gas supplying unit (16) that is capable of supplying the gas to the culturing unit, and the guide unit (30a to 30d, 92) that guides the gas into the culturing solution, while in addition, the gas is supplied from the gas supplying unit to the guide unit, and the culturing solution is drawn in from the exterior of the guide unit into the interior (42) of the guide unit through the suction port (44) of the guide unit, and further, the convection is caused to be generated in the culturing solution by discharging the culturing solution from the interior of the guide unit through a discharge port (46) of the guide unit.

In the present embodiment, the culturing device is disclosed, in which the convection generating unit comprises the gas supplying unit (16) that is capable of supplying the gas to the culturing unit, and the guide unit (30a to 30d, 92) that guides the gas into the culturing solution, the guide unit comprises the suction port (44) that draws in the culturing solution from the exterior of the guide unit into the interior (42) of the guide unit, and the discharge port (46) that discharges the culturing solution from the interior of the guide unit to the exterior of the guide unit, and the convection generating unit supplies the gas from the gas supplying unit to the guide unit, and by drawing in the culturing solution from the suction port of the guide unit, and by discharging the culturing solution from the discharge port of the guide unit, causes the convection to be generated in the culturing solution.

In accordance with such a configuration, by supplying the gas to the guide unit, the convection can be made to be generated in the culturing solution. Accordingly, there is no need to provide a stirring blade or a motor or the like in order to cause the convection to be generated. Consequently, while the culturing device is simplified, the microalgae can be satisfactorily and easily agitated.

In the present embodiment, the culturing method is disclosed, in which the discharge port of the guide unit is oriented in the predetermined direction (X2) that intersects with respect to the vertical direction of the accommodation unit, and the culturing solution is discharged in the predetermined direction through the discharge port.

In the present embodiment, the culturing device is disclosed, in which the discharge port of the guide unit is oriented in the predetermined direction (X2) that intersects with respect to the vertical direction of the accommodation unit, and the culturing solution is discharged in the predetermined direction through the discharge port.

Since the culturing solution is discharged from the discharge port that faces in the predetermined direction, in the culturing solution, it becomes easy to generate the convection so as to flow in the predetermined direction. Accordingly, it becomes easy for the microalgae that are suspended by the culturing solution that is discharged to flow along with the convection. As a result, the microalgae are more satisfactorily agitated.

In the present embodiment, the culturing method is disclosed, in which the collection pipe includes the collection port (56) in order to collect the culturing solution, the collection port is oriented in a direction (X1) opposite to the predetermine direction in which the discharge port of the guide unit is oriented, and the culturing solution, which is discharged in the predetermined direction through the discharge port, is drawn in by the collection port.

In the present embodiment, the culturing device is disclosed, in which the collection pipe includes the collection port (56) in order to collect the culturing solution, the collection port is oriented in a direction (X1) opposite to the predetermine direction in which the discharge port of the guide unit is oriented, and the culturing solution, which is discharged in the predetermined direction through the discharge port, is drawn in by the collection port.

In the collection pipe, the culturing solution is discharged from the collection port that faces in the direction opposite to the predetermined direction.

Therefore, it becomes easy for the microalgae that are suspended by the culturing solution that is discharged to flow along with the convection. As a result, the microalgae are even more satisfactorily agitated.

In the present embodiment, the culturing method is disclosed, in which the culturing device comprises a single individual collection pipe (94), and the guide unit is arranged in the accommodation unit at one end part (X1) in the widthwise direction (X) that intersects with respect to the vertical direction (Z), and the single individual collection pipe is arranged at another end part (X2) in the widthwise direction.

In the present embodiment, the culturing device (90) is disclosed, in which the culturing device comprises a single individual collection pipe (94), and the guide unit is arranged in the accommodation unit at one end part (X1) in a widthwise direction (X) that intersects with respect to a vertical direction (Z), and the single individual collection pipe is arranged at another end part (X2) in the widthwise direction.

In this case, the microalgae that have settled in the corners of the accommodation unit where convection is unlikely to occur are suspended by the guide unit and the collection pipe, and thereafter, it becomes easy for the microalgae to flow due to the convection. Accordingly, the microalgae and the culturing solution are agitated throughout the entirety thereof.

In the present embodiment, the culturing method is disclosed, in which the culturing device comprises the plurality of individual guide units (30a to 30d) including the guide unit, and the plurality of individual collection pipes (32a to 32d) including the collection pipe, and an individual one (30a) from among the plurality of individual guide units is arranged in the accommodation unit at one end part in the widthwise direction (X) intersecting the vertical direction (Z), and an individual one (32d) from among the plurality of individual collection pipes is arranged at another end part (X2) in the widthwise direction, the remaining ones (30b to 30d) from among the plurality of individual guide units are arranged between the guide unit (30a) arranged at the one end part and the collection pipe (32d) arranged at the other end part, and the remaining ones (32b to 32d) from among the plurality of individual collection pipes are arranged respectively adjacent to the remaining one from among the plurality individual guide units.

In the present embodiment, the culturing device is disclosed, in which the culturing device comprises the plurality of individual guide units (30a to 30d) including the guide unit, and the plurality of individual collection pipes (32a to 32d) including the collection pipe, and an individual one (30a) from among the plurality of individual guide units is arranged in the accommodation unit at one end part in the widthwise direction (X) intersecting the vertical direction (Z), and an individual one (32d) from among the plurality of individual collection pipes is arranged at another end part (X2) in the widthwise direction, the remaining ones (30b to 30d) from among the plurality of individual guide units are arranged between the guide unit (30a) arranged at the one end part and the collection pipe (32d) arranged at the other end part, and the remaining ones (32b to 32d) from among the plurality of individual collection pipes are arranged respectively adjacent to the remaining one from among the plurality individual guide units.

In this case, a small amount of convection occurs between the one individual guide unit and the one individual collection pipe that faces toward the guide unit. Further, by the small amounts of convection being combined, a large amount of convection is generated over the entirety of the widthwise direction of the accommodation unit. By the small amounts of convection and the large amount of convection, the microalgae and the culturing solution are capable of being effectively agitated.

It should be understood that the present invention is not limited to the embodiment described above, and various additional or modified configurations could be adopted therein without departing from the essence and gist of the present invention.

What is claimed is:

1. A culturing method for culturing microalgae in a culturing device equipped with an accommodation unit in which a culturing solution and the microalgae are accommodated, and a gas supplying unit configured to supply a gas to the accommodation unit, wherein:
in the accommodation unit, there are provided a convection generating unit configured to cause convection to be generated in the culturing solution, and a collection pipe configured to collect the culturing solution from the accommodation unit; and
a pump configured to be capable of drawing in and discharging the culturing solution through the collection pipe;
wherein, during culturing of the microalgae, and while causing convection to be generated in the culturing solution in the convection generating unit, the culturing solution is collected through the collection pipe by the pump, and thereafter, the collected culturing solution is discharged through the collection pipe by the pump.

2. The culturing method according to claim 1, wherein:
the convection generating unit comprises a gas supplying unit configured to be capable of supplying gas to the culturing unit, and a guide unit configured to guide the gas into the culturing solution;
the gas is supplied from the gas supplying unit to the guide unit, and the culturing solution is drawn in from the exterior of the guide unit into the interior of the guide unit through a suction port of the guide unit, and convection is caused to be generated in the culturing solution by discharging the culturing solution from the interior of the guide unit to the exterior of the guide unit through a discharge port of the guide unit.

3. The culturing method according to claim 2, wherein the discharge port of the guide unit is oriented in a predetermined direction that intersects with respect to a vertical direction of the accommodation unit, and the culturing solution is discharged in the predetermined direction through the discharge port.

4. The culturing method according to claim 3, wherein the collection pipe includes a collection port in order to collect the culturing solution, the collection port is oriented in a direction opposite to the predetermine direction in which the discharge port of the guide unit is oriented, and the culturing solution that is discharged in the predetermined direction through the discharge port is drawn in by the collection port.

5. The culturing method according to claim 3, wherein
the culturing device comprises a single individual collection pipe; and
the guide unit is arranged in the accommodation unit at one end part in a widthwise direction that intersects with respect to a vertical direction, and the single individual collection pipe is arranged at another end part in the widthwise direction.

6. The culturing method according to claim 3, wherein
the culturing device comprises a plurality of individual guide units including the guide unit, and a plurality of individual collection pipes including the collection pipe; and
an individual one from among the plurality of individual guide units is arranged in the accommodation unit at one end part in a widthwise direction intersecting a vertical direction, and an individual one from among the plurality of individual collection pipes is arranged at another end part in the widthwise direction;
remaining ones from among the plurality of individual guide units are arranged between the guide unit arranged at the one end part and the collection pipe arranged at another end part; and
remaining ones from among the plurality of individual collection pipes are arranged respectively adjacent to the remaining ones from among the plurality individual guide units.

7. A culturing device configured to culture microalgae in a culturing solution, the culturing device comprising:
an accommodation unit in which the culturing solution and the microalgae are accommodated;
a convection generating unit provided in the accommodation unit and configured to cause convection to be generated in the culturing solution;
a collection pipe provided in the accommodation unit and configured to collect the culturing solution from the accommodation unit;
a pump configured to be capable of drawing in and discharging the culturing solution through the collection pipe; and
a control unit configured to control the pump;
wherein, during culturing of the microalgae and while causing convection to be generated in the culturing solution in the convection generating unit, the control unit performs a control to collect the culturing solution through the collection pipe by the pump, and thereafter, to discharge the collected culturing solution through the collection pipe by the pump.

8. The culturing device according to claim 7, wherein:
the convection generating unit comprises a gas supplying unit configured to be capable of supplying gas to the culturing unit, and a guide unit configured to guide the gas into the culturing solution;
the guide unit comprises a suction port configured to draw in the culturing solution from the exterior of the guide unit into the interior of the guide unit, and a discharge port configured to discharge the culturing solution from the interior of the guide unit to the exterior of the guide unit; and
the convection generating unit supplies the gas from the gas supplying unit to the guide unit, and by drawing in the culturing solution from the suction port of the guide unit and by discharging the culturing solution from the discharge port of the guide unit, causes the convection to be generated in the culturing solution.

9. The culturing device according to claim 8, wherein the discharge port of the guide unit is oriented in a predetermined direction that intersects with respect to a vertical direction of the accommodation unit, and the guide unit discharges the culturing solution in the predetermined direction through the discharge port.

10. The culturing device according to claim 9, wherein the collection pipe includes a collection port in order to collect the culturing solution, the collection port is oriented in a direction opposite to the predetermine direction in which the discharge port of the guide unit is oriented, and the culturing solution that is discharged in the predetermined direction through the discharge port is drawn in by the collection port.

11. The culturing device according to claim 9, wherein
the culturing device comprises a single individual collection pipe; and
the guide unit is arranged in the accommodation unit at one end part in a widthwise direction that intersects with respect to a vertical direction, and the single individual collection pipe is arranged at another end part in the widthwise direction.

12. The culturing device according to claim 9, wherein
the culturing device comprises a plurality of individual guide units including the guide unit, and a plurality of individual collection pipes including the collection pipe; and
an individual one from among the plurality of individual guide units is arranged in the accommodation unit at one end part in a widthwise direction intersecting a vertical direction, and an individual one from among the plurality of individual collection pipes is arranged at another end part in the widthwise direction;
remaining ones from among the plurality of individual guide units are arranged between the guide unit arranged at the one end part and the collection pipe arranged at another end part; and
remaining ones from among the plurality of individual collection pipes are arranged respectively adjacent to the remaining ones from among the plurality individual guide units.
